(19) United States
(12) Patent Application Publication
(10) Pub. No.: US 2008/0004520 A1
(43) Pub. Date: Jan. 3, 2008

(54) System for and method of diagnostic coding using medical image data

(76) Inventor: Richard H. Theriault, Lincoln, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 11/818,329

(22) Filed: Jun. 14, 2007

Related U.S. Application Data
(60) Provisional application No. 60/813,908, filed on Jun. 15, 2006.
(60) Provisional application No. 60/813,909, filed on Jun. 15, 2006.
(60) Provisional application No. 60/813,907, filed on Jun. 15, 2006.
(60) Provisional application No. 60/813,844, filed on Jun. 15, 2006.

(51) Int. Cl. A61B 5/055

(52) U.S. Cl. 600/410

(57) ABSTRACT

In accordance with at least one aspect, a method is provided to automatically generate a diagnosis based on information provided in a subject MRI image. In one embodiment, a plurality of stored MRI images are associated with one or more pathological conditions. A region of interest is identified in a subject color MRI image. A closest match between the subject MRI image and a stored image is determined by comparing the region of interest to a region of at least one of the plurality of stored images. A diagnosis is generated for the subject image.

### RELATED APPLICATIONS

This application claims the benefit under 35 U.S.C. § 119(e) to each of the following co-pending U.S. provisional patent applications: Ser. No. 60/813,908 entitled “System For and Method of Performing a Medical Diagnosis,” filed Jun. 15, 2006; Ser. No. 60/813,909 entitled “System for and Method of Diagnostic Coding Using Medical Image Data,” filed Jun. 15, 2006; Ser. No. 60/813,907 entitled “System For and Method of Increasing the Efficiency of a Diagnostic Review of Medical Images,” filed Jun. 15, 2006; and Ser. No. 60/813,844, entitled “Three-Dimensional Rendering of MRI Results Using Automatic Segmentation,” filed on Jun. 15, 2006, each of which is hereby incorporated herein by reference in its entirety. This application is also related to the patent applications entitled: “System for and Method of Diagnostic Review of Medical Images,” Attorney Docket No. C2046-700110; “Three Dimensional Rendering of MRI Results Using Automatic Segmentation” Attorney Docket No. C2046-700210; and “System for and Method of Performing a Medical Evaluation,” Attorney Docket No. C2046-700310; each of which has Richard H. Theriault as inventor and filed on even date herewith and each of which is hereby incorporated herein by reference in its entirety.

### BACKGROUND OF INVENTION

1. Field of Invention

Embodiments of the invention relate generally to medical imaging. More specifically, at least one embodiment relates to a system and method for employing color magnetic resonance imaging technology for medical evaluation, diagnosis and/or treatment.

2. Discussion of Related Art

Today, doctors and others in the health care field rely heavily on magnetic resonance imaging (“MRI”) technology when assessing the health of patients and possible courses of treatment. Current diagnostic procedures sometimes employ a comparison between a current image from a patient who is being diagnosed and prior images from other patients. For example, the current image may include a particular organ and/or region of the body which may include evidence of a pathological condition (e.g., a diseased organ). Generally, abnormalities are reflected in such images because they contain a non-typical pattern (i.e., non-typical of a healthy subject) formed by shading in the image. In such a case, the prior images may be of the same organ and/or region of the body from the prior patients who suffered from a positively identified abnormality. Historically, healthcare professionals performed diagnosis by referring to bound sets of such images to try to locate a prior image that illustrates a pattern similar to the pattern in the suspect region of the current image (i.e., the image being evaluated for diagnosis). A close match provides the healthcare professional with an indication that the current image is illustrative of the same or similar abnormality.

However, accurate diagnosis and analysis performed using MRI images is nuanced and takes considerable experience. In particular, it is often difficult and time consuming for a professional to reach a conclusion that an image or a set of images is “normal” with a high degree of confidence. That is, it may be difficult to determine with a high degree of confidence that an image does not include a physiological abnormality. The preceding situation is in part the result of the desire to eliminate false negatives. For example, where MRI images are employed to screen for a life threatening disease, there is a risk of potentially fatal consequences if a clean bill of health is mistakenly provided as a result of a review of a set of MRI images when the disease is actually present but perhaps difficult to identify from the images.

The above-described situation is made more difficult because the number of radiologists and other experienced professionals qualified to perform diagnostic review of medical images is decreasing while the volume of images continues to grow.

There have been attempts to provide dataset matching using software that matches a current image with a stored image based on the data provided by the values of gray-scale pixels included in two images that are compared. However, gray-scale images do not provide or convey nearly as much information as a color image. Also, it is tedious and time consuming to build a database of images for comparison because there are no effective processes to automatically segment gray-scale images.

Further, the utility of current systems is limited because they do not provide any diagnostic coding information to the healthcare professional. Diagnostic coding information includes information indicative of the characteristics, class, type, etc. of an abnormality. Thus, current methods do not provide the preceding information concerning the results of a comparison (and a possible match) between a reference image and the current image. As a result, current systems require that the healthcare professional manually compare the “matching” image and the current image to make a diagnostic evaluation.

Various approaches have been developed in an effort to improve the diagnostic-accuracy and diagnostic-utility of information provided by a set of MRI images. In one approach, color images are generated to provide a more realistic appearance that may provide more information than the information provided in gray-scale images. For example, intensity is the only variable for pixels in a gray-scale image. Conversely, each pixel in a color image may provide information based on any or all of the hue, saturation and intensity of the color of the pixel. One such approach is described in U.S. Pat. No. 5,332,968, entitled “Magnetic Resonance Imaging Color Composites,” issued Jul. 26, 1994, to Hugh K. Brown (“the '968 patent”) which describes the generation of composite color MRI images from a plurality of is MRI images. The '968 patent is incorporated herein by reference in its entirety.

The term “slice” is used herein to refer to a two dimensional image generally. The term “slice” is not intended to describe a specific image format and a slice may be in any of a variety of image formats and/or file-types, including MRI and CT images, TIFF and JPEG file-types.

The '968 patent describes that a plurality of slices which are two dimensional images (e.g., MRI images) may be captured where each slice is based on different image acquisition parameters. As is well known in the art, in one approach, a first slice may be generated using a T1-weighted process, a second slice may be generated using a T2-weighted process, and a third slice may be generated using a proton-density weighted process. The '968 patent describes a process whereby a composite image having a semi-natural anatomic appearance is formed from the slices that are associated with the same region of the object that is scanned. However, the approaches described in the '968 patent fail to consider that, in practice, the slices captured with the various parameters do not precisely align because, for example, they are not captured at precisely the same point in time. The result is that the composite image includes some inaccuracies at the boundaries between different regions in the image. This limits the diagnostic value of the composite color images described in the '968 patent because the health care professional must still manually review the images to more precisely determine the locations of various objects, for example, the location of region boundaries in the image, the locations of organs in images of the human body, etc. That is, current approaches require human review to establish boundaries of object and/or regions in the images such as regions of the human anatomy that may or may not be diseased. The preceding is particularly problematic where the information in the image is used for surgical planning.

### SUMMARY OF INVENTION

In one aspect of the invention, a method is provided for automatically generating a diagnosis based on information provided in the subject MRI image. According to one embodiment, the method includes an act of associating each of a plurality of reference color MRI images corresponding to one or more pathological conditions with a diagnosis, respectively; identifying a region of interest in the subject MRI image; comparing the region of interest to one or more regions of at least one of the plurality of reference color MRI images; determining a closest match between the subject MRI image and a reference image selected from among the plurality of reference color MRI images; and generating a diagnosis associated with a subject MRI image based at least partly on a pathological condition associated with the reference image. In accordance with one embodiment, the method also includes an act of assigning a confidence factor to the diagnosis. In a further embodiment, the method also includes an act of assigning a diagnosis code to the subject image where the diagnosis code corresponds to the generated diagnosis. In yet another embodiment the method includes the act of determining a strength of the closest match.

In another aspect, the invention provides a system for generating a diagnostic code based on information provided in a subject MRI image. In according with one embodiment the system includes a colorization module, a reference image storage module, a processing module, and a coding module. In one embodiment, the colorization is adapted to generate the subject MRI image in color by generating a composite color image from a plurality of grey-scale images. In a further embodiment, the reference image storage module is adapted to store a plurality of color reference MRI images that include at least one reference image having a region in which a know pathological condition is present. In one embodiment, the processing module is adapted to compare the subject image with the at least one reference image. In yet a further embodiment, the coding module is adapted to generate a diagnostic code concerning the subject image based on a comparison between the information provided in the subject MRI image and information provided in the at least one reference image.

In various embodiments of the system, the information provided in the at least one reference image may be information concerning the pathological condition. In addition, the information concerning the pathological condition may be information concerning the region in which the known pathological condition is represented. In one embodiment the information concerning the pathological condition is provided by color that is present in the region. In accordance with one embodiment, the coding module is adapted to generate the diagnostic code based on a comparison between the information provided in the subject MRI image and information provided in a plurality of reference images.

### BRIEF DESCRIPTION OF DRAWINGS

The patent or application file contains at least one drawing executed in color. Copies of this patent or patent application publication with color drawings will be provided by the Office upon request and payment of the necessary fee.

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a system for processing color MRI images for diagnostic analysis in accordance with one embodiment of the invention;

FIG. 2 illustrates a display that includes a plurality of sets of medical images including a set of composite color images in accordance with an embodiment of the invention;

FIG. 3 illustrates a display that includes the composite color images of FIG. 2 in accordance with an embodiment of the invention;

FIG. 4 illustrates a single image selected from the composite color images of FIG. 3 in accordance with one embodiment of the invention;

FIG. 5 illustrates a display including a color composite image in accordance with an embodiment of the invention;

FIG. 6A illustrates a system for processing reference images in accordance with an embodiment of the invention;

FIG. 6B illustrates an image database in accordance with one embodiment of the invention;

FIG. 7 illustrates a process in accordance with an embodiment of the invention;

FIG. 8 illustrates a block diagram of a system for processing color MRI images for diagnostic analysis in accordance with an embodiment of the invention;

FIG. 9 illustrates a block diagram of a computer system for embodying various aspects of the invention; and

FIG. 10 illustrates a storage sub system of the computer system of FIG. 9 in accordance with an embodiment of the invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

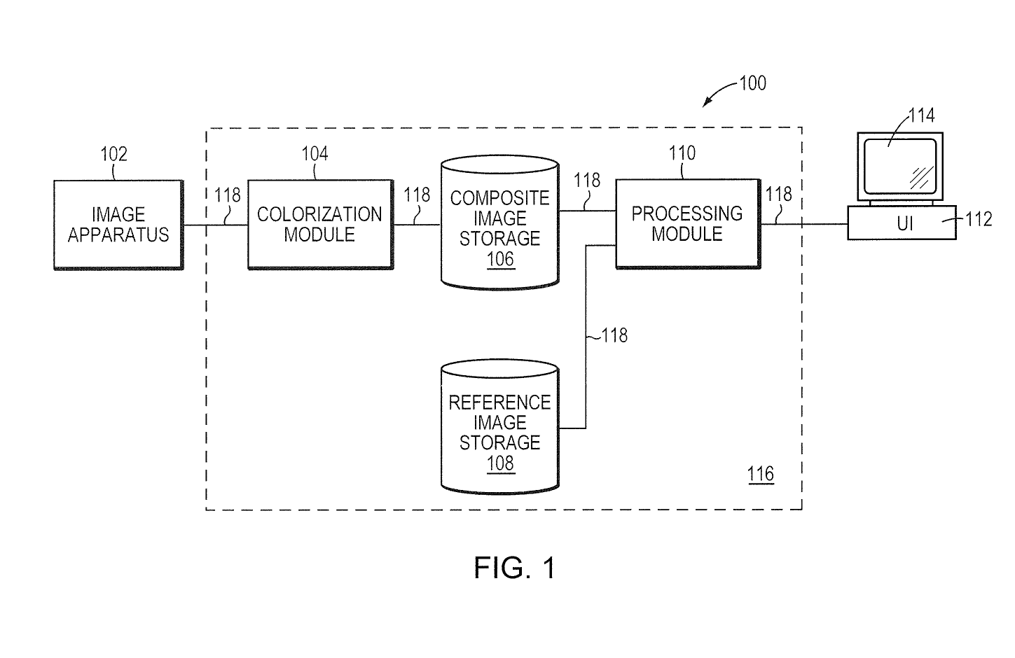

Referring to FIG. 1, a system for processing color MRI images for diagnostic analysis is illustrated. The system 100 includes image generation apparatus 102, colorization module 104, a composite image storage module 106, a reference image storage module 108, a processing module 110 and a user interface 112. The image generation apparatus 102 may be any of those apparatus that are well know by those of ordinary skill in the art. In one embodiment, the system 100 may be used in the health care field and the image generating apparatus 102 may, for example, include one or more of a MRI image generating apparatus, computed tomography (“CT”) image generating apparatus, ultrasound image generating apparatus, and the like. In one embodiment the image generating apparatus is an MRI unit, for example, a GE MEDICAL SIGNA HD SERIES MRI or a SIEMENS MEDICAL MAGNATOM SERIES MRI.

The colorization module 104 is employed to produce colored images from the images that are generated from the image generating apparatus, for example, as described in the '968 patent. In one embodiment, the processes described in the '968 patent provides a color coefficient to generate images using additive RGB color combinations. In various embodiments, the colorization module may employ either automatic colorization processes and/or manual colorization processes. For example, in one embodiment quantitative data supplied by the gray tone images generated by the image generating apparatus 102 is reviewed by an operator in order to assign the color coefficients. In some embodiments the color coefficients are established to highlight one or more biological substances and/or anatomical structures. In particular, the separate images (e.g., slices) of a common region collected using the different image generating parameters may be particularly well suited to identify a specific tissue or anatomical structure. In one example provided in the '968 patent, follicular fluid is co-dominant in the T2-weighted and proton density images while fat is co-dominant in the T1 and proton density weighted images, and muscle is slightly dominant in the proton density image when compared to the T1 and T2-weighted images.

Accordingly, in one embodiment, a color palette may be selected to highlight a first physical attribute (e.g., fat content, water content or muscle content) in a first color and highlight a second physical attribute in a second color. As is described in further detail herein, the color selection/assignment results in the generation of composite colors when multiple images are combined. Further the composite colors may have increased diagnostic value as compared to the original color images.

The colorization module 104 may be implemented in hardware or software and in one embodiment is a software module. In other embodiments, the colorization module includes a plurality of software modules, for example, a first module that generates monochrome images based on color coefficients and pixel values and a second software module that generates a composite image that accounts for the information provided in each of the monochrome images. In various embodiments, the operator may employ the user interface 112 to operate the colorization module 104 and complete the colorization process and generation of a composite color image. However, in some embodiments the operator may use a user interface that is located elsewhere in the system 100 to access and control the colorization module.

In one approach, the color assignment may be determined using the value of the Hounsfield unit for various types of tissues. According to one embodiment, the color assignment is automatically determined by determining the Hounsfield unit for a pixel and then assigning the color intensity for the pixel based on a value of the Hounsfield unit for that pixel.

In accordance with one embodiment, once the composite image is generated it can be stored in the composite image storage module 106. The composite image storage module 106 may be implemented in any of a variety of manners that are well known by those of ordinary skill in the art. For example, the composite image storage module may be an image database which stores the images in an electronic format on a computer storage medium including RAM or ROM. The image database may include well known database systems such as those offered by Oracle Corporation. In addition, in one embodiment, the composite image storage module 106 may store color images generated by any means, for example, the images may not be “composite” images.

The system 100 also includes the reference image storage module 108 which may include a plurality of reference images including color reference images and composite color reference images that were previously generated. These reference images may include images that illustrate one or a plurality of abnormalities. As a result, the reference images may be used for comparison purposes with a current image which is undergoing diagnosis for a potential abnormality (e.g., for detection of a pathological condition). In some embodiments, the reference images also include images that illustrate healthy subjects and do not include any abnormalities.

In the illustrated embodiment, the system 100 also includes a processing module 110 which may be employed to perform the comparison between the current image supplied from the composite image storage module and one or more reference images in order to provide analysis and diagnostics. The processing module 110 may also be implemented in hardware, software, firmware or a combination of any of the preceding. In various embodiments, the processing module 110 can operate automatically to compare a composite image (including a newly-generated image) with one or a plurality of reference images to determine whether an abnormality exists. In addition, the user interface 112 may be employed by a healthcare professional to view and compare the current composite image, one or more reference images and/or to review results of a diagnostic comparison of two or more images.

In one embodiment, the user interface 112 may include a display 114 such as a CRT, plasma display or other device capable of displaying the images. In various embodiments, the display 114 may be associated with a user interface 112 that is a computer, for example, a desktop, a notebook, laptop, hand-held or other computing device that provides a user an ability to connect to some or all of the system 100 in order to view and/or manipulate the image data that is collected and/or stored there.

In accordance with one embodiment, in addition to the ability to perform various comparisons of current images and stored reference images for diagnostic purposes, the processing module 110 may also be employed to perform additional manipulation of the colorized images and the information provided therein. In general, the processing module 110 may be employed in the system 100 to perform a variety of functions including the registration of a plurality of slices captured by the image generating apparatus 102, the segmentation of one or more images as a result of the information provided by the image, and the generation of three-dimensional (“3D”) composite images.

In one embodiment, one or more of the colorization module 104, the composite image storage 106, the reference image storage 108, and the processing module 110 are included in a computer 116. Other configurations that include a plurality of computers connected via a network 118 may also be employed. For example, the processing module 110 may be included in a first computer while others of the preceding modules and storage are included in one or more additional computers. In another embodiment, the processing module 110 is included in a computer with any combination of one or more of the colorization module 104, the composite image storage 106, and the reference image storage 108.

The overall process of capturing a set of MRI images is described here at a high level to provide some background for the material that follows. The following description is primarily directed to MRI analysis performed on a human subject, however, the imaging system may be any type of imaging system and in particular any type of medical imaging system. In addition, the following processes may be employed on subjects other than human subjects, for example, other animals or any other organism, living or dead.

In general, a multi-parameter analysis is performed to capture two-dimensional slices of a subject of the MRI analysis. If for example, the chest cavity is the subject of the imaging, a series of two-dimensional images are created by, for example, capturing data on a series of slices that are images representative of an x-y plane oriented perpendicular to the vertical axis of the subject. For example, where the subject is a human, a z-axis may be identified as the axis that runs from head to toe. In this example, each slice is a plane in an x-y axis extending perpendicular to the z-axis, e.g., centered about the z-axis. As a result, an MRI study of a subject's chest may include a first image that captures the anatomy of the subject in a plane. In one embodiment, following a small gap (i.e., a predetermined distance along the z-axis), a second image is created adjacent the first image in a direction toward the subject's feet. The process is repeated for a particular set of image-generating parameters (e.g., T1-weighted, T2-weighted, PD-weighted, etc.) until the section of the subject's anatomy that is of interest is captured by a set of images using the first image parameters. A second set of images may subsequently be generated using a second set of image-generating parameters. In one embodiment, other additional sets of images each with the same plurality of slices may also be generated in like fashion. The determination of the region to be examined using the image generating apparatus and the various image generating parameters to be used are generally determined (e.g., by a healthcare professional) in advance of the subject undergoing the imaging. As a result, a plurality of sets of images each including a plurality of slices may be created for the subject.

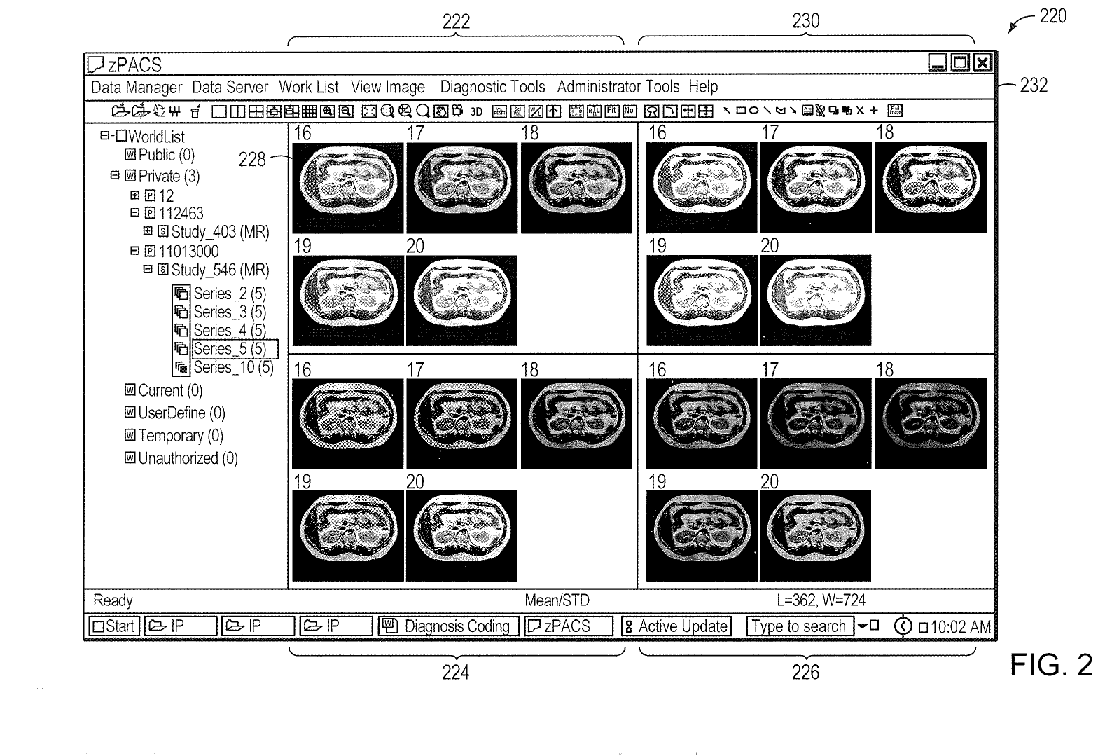

Referring now to FIG. 2, a display 220 includes a plurality of sets of MRI images in accordance with one embodiment. FIG. 2 includes a first set 222 of gray-scale images produced using a first set of parameters, a second set 224 of gray-scale images produced using a second set of parameters and a third set 226 of gray-scale images produced using a third set of parameters. Because different image generating parameters are used to create each of the sets, the gray-scale intensity of various regions may differ for the same portion of the anatomy from set to set. For example, the lungs may appear with a first gray-scale intensity in set 1 and a second gray-scale intensity in set 2.

Each of the sets also includes a plurality of slices 228 in the illustrated embodiment. Each of the sets 222, 224, 226 includes five images (i.e., “slices”) identified as 16, 17, 18, 19 and 20. In accordance with one embodiment, each slice is an image of a plane and/or cross-section of the subject. The slices in each set correspond to the slices of each of the other sets that are identified with the same number. As mentioned previously, however, the alignment of the slices is such that they may not be of the exact or precisely the identical region.

A fourth set 230 of slices 232 is also illustrated in the display 220. The fourth set 230 is a composite colorized set of images corresponding to the slices 16, 17, 18, 19 and 20. According to one embodiment, the image generating apparatus 102 of the system 100 generates each of the slices 16-20 of the first set 222, the second set 224, and the third set 226, respectively. The colorization module 104 then combines the data provided by the slices in each set to generate the composite color slices in the fourth set 230. For example, the data from slice 16 of the first set 222, slice 16 of the second set 224 and slice 16 of the third set 226 are employed to generate slice 16 of the fourth set. A similar approach is employed to generate each of the remaining composite color slices in the fourth set 224. The sets of five slices provide a simplified example for purposes of explanation. In general, actual MRI studies may include a much greater quantity of slices.

In addition, in various embodiments, each of the sets 222, 224 and 226 may be stored temporarily or permanently in memory included in the image generating apparatus 102, or in a database elsewhere in the system 100, for example, in a database that also includes either or both of the composite image storage 106 and the reference image storage 108.

As mentioned previously, approaches to generating composite color MRI images are generally familiar to those of ordinary skill in the art. However, improved processes are necessary to increase the diagnostic utility of color images and in particular, to provide information in a form that is more accurately interpreted by computer systems, e.g., automatically interpreted.

Accordingly, embodiments of the invention, apply segmentation processes to more precisely distinguish different regions within each of the composite color images. In one embodiment, a segmentation process achieves accuracy to within plus or minus several millimeters within a single slice. In a version of this embodiment, the segmentation process accurately identifies boundaries between different regions in a slice to within ±5 mm or less. In another version, the segmentation process accurately identifies boundaries between different regions in a slice to within ±3 mm or less. In various embodiments, the segmentation process is performed automatically. That is, the segmentation process is performed on an image without any manual oversight yet achieves the preceding or greater accuracy without the need for post-processing review, e.g., without the need for a human to review and refine the results.

In the medical field, an exemplary list of the various different regions that can be distinguished include: regions of healthy tissue distinguished from regions of unhealthy tissue; a region of a first organ distinguished from a region of a second organ; an organ distinguished from another part of the anatomy; a first substance (e.g., blood that is freshly pooled) from a second substance (e.g., “dried blood” from a pre-existing condition); a first region having a first ratio of fat to water and a second region having a second ration of fat to water, etc.

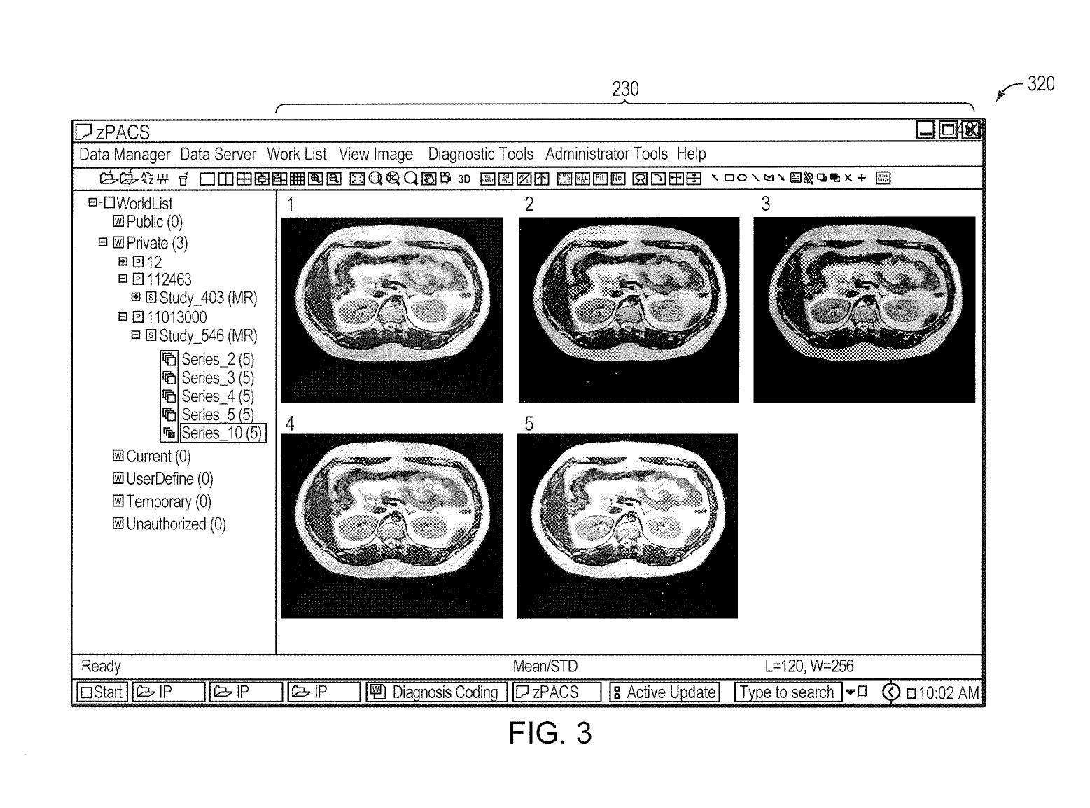

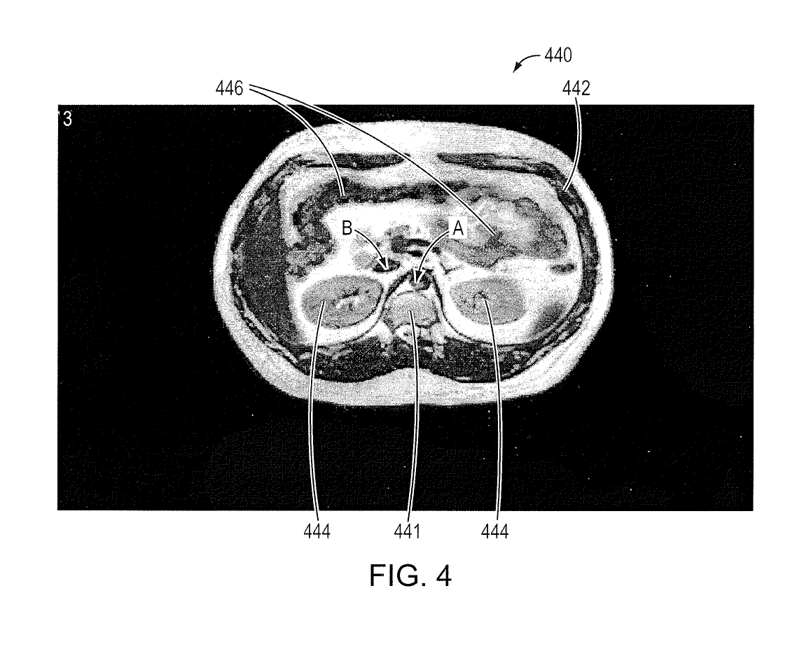

FIGS. 3 and 4 include one or more of the slices from the fourth set 230, however, the slices 16, 17, 18, 19 and 20 are renumbered 1, 2, 3, 4 and 5, respectively. Referring to FIG. 3, in one embodiment, a display 320 includes the fourth set 230 of slices 232 magnified relative to their appearance in FIG. 2. FIG. 4 includes an image 400 of a single slice, slice 3 (i.e., slice 18), from the fourth set 230 further magnified relative to both FIGS. 2 and 3. The illustrated slice 3 is an image of a portion of the abdominal region of a patient. Among other portions of the anatomy, the spine 441, the rib cage 442, the kidneys 444, and the intestines 446 appear distinctly in the composite color image of the slice 18.

Upon inspection, it is also apparent that a yellowish/red region A appears at the center of the slice while the red region B appears without any yellow color component to the left center of the image. In accordance with one embodiment, the difference in color between these two regions may be medically important, and in particular, may provide information concerning a pathological condition of the subject. In one version, the difference in color indicates that the region A may include dried blood. In another example, a composite color may result that is indicative of the freshness of blood where “new” blood may be an indication that an internal injury (e.g., a brain contusion) is actively bleeding.

Further, a particular composite color may be established as representative of a particular region in various embodiments, e.g., associated with a particular type of tissue. Accordingly, a user may establish a color palette for the various physical parameters appearing in a set of images (e.g., water, fat, muscle, etc.) such that the selected color is associated with the region-type selected by the user in the composite color image. As another example, where a composite color is representative of a ratio of fat to water in a region, the shade and/or intensity of that particular color may be useful in diagnosing whether or not a tumor is malignant because the fat-to-water ratio may be indicative of a malignancy.

In general, the distinction between the appearance of region A and region B results in the identification of a region of interest (“ROI”) that may be examined more closely and/or compared with regions from previous MRI studies that may illustrate various pathological conditions. For example, the ROI may be compared with images and regions of images from other patients where the image includes an identified abnormality (e.g., pathological condition) indicative of injury, disease, and/or trauma.

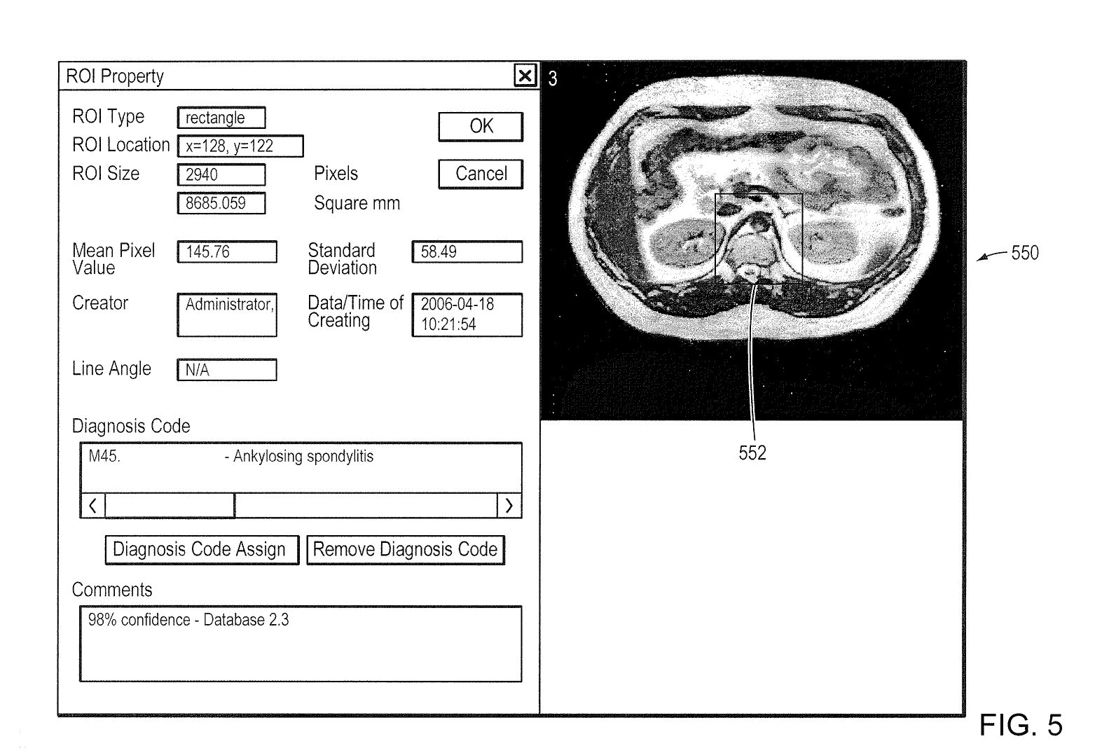

In various embodiments of the invention, such ROIs may be automatically identified using one or more software modules. FIG. 5 illustrates a display 550 in which a ROI 552 (including region A) within slice 18 of the fourth set 230 is identified.

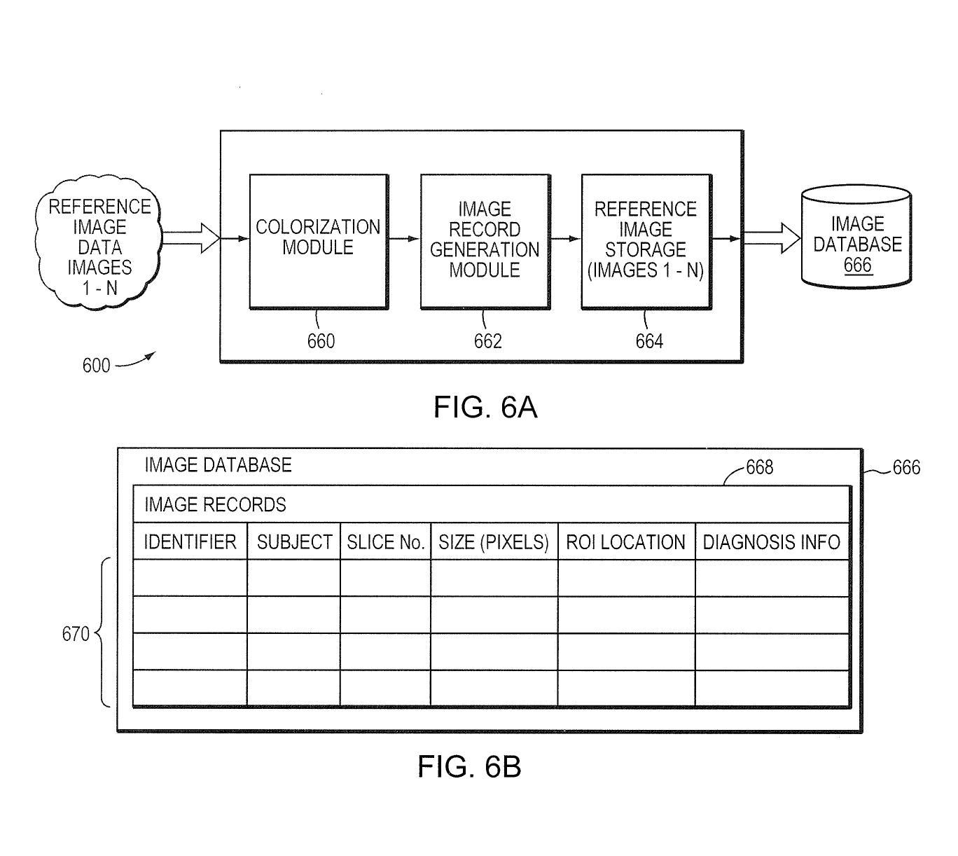

In accordance with one or more embodiments, the processing module 110 of the system 100 may perform comparisons between a current image undergoing diagnostic analysis and one or more reference images 108. As illustrated in FIG. 6, in accordance with one embodiment, a system 600 can be employed to process a plurality of reference images that may be used for comparison. In one embodiment, the system 600 can be included as an element of the system 100. In a further embodiment, the system 600 is included in a processing module (e.g., the processing module 110). In another embodiment, the system 600 is included in the reference image storage module 108 of the system 100.

In various embodiments, the overall operation of the system 600 may include any of the following processes alone or in combination with any of the listed processes or in combination with other processes, the processes may include: the generation of composite color images; the generation of an image record associated with each image; and the storage of the images.

In accordance with one embodiment, the system 600 may include a colorization module 660, an image record generation module 662 and a reference image storage module 664. In addition, the system 600 may also include an image database 666.

In one embodiment, the system 600 receives reference image data for a plurality of images (e.g., images 1-N) that may have been previously generated as a result of MRI studies performed on one or more previous patients. In accordance with one embodiment, the images include abnormalities (e.g., pathological conditions). In various embodiments, the system 600 converts the reference images into a format that may be processed by, for example, the processing module 110 of the system 100 and storing the reference images in a manner that they are easily identifiable and retrievable for later processing by the system 100. For example, the system 600 converts the reference images into a format that is useful in performing comparisons/analysis of subject images with the reference images.

In accordance with one embodiment, the colorization module 660 employs any of the approaches known to those of ordinary skill in the art for generating a composite color image from one or more slices that are generated in the MRI study. For example, in one version, the colorization processes described in the '968 patent may be employed.

In one embodiment, the image record generation module 662 assigns identifying and diagnostic information to each image. In a version of this embodiment, the image record generation module is included as part of the colorization process and is performed by the colorization module, while in other alternate embodiments, the image record generation module 662 generates an image record either subsequent to or prior to the processing by the colorization module 660. As a result, each of the reference images may be stored by the reference image storage module 664 in association with the image record, for later retrieval. The image database may be located as an integral part of the system 600 or may be a separate device. The image database 666 may include only reference images. However, in another embodiment the image database employed for storage of reference image data is also used to store composite images of the subject patient or patients.

In various embodiments, the image database may be included at a central host server accessible over a network, for example, a local area network (LAN) or a wide area network (WAN), for example, the Internet.

Referring now to FIG. 6B, the image database includes image records 668 for a plurality of images 670 or each image is associated with an identifier, a subject, a slice number, a size, the location of a region of interest, and diagnostic information.

In accordance with one embodiment, the identifier is a unique number that is assigned an image so that it may be later retrieved based on the positive identification provided by the identifier. The identifier may include alpha, numeric, or alpha-numeric information.

In one embodiment, the subject field may be used to identify a particular part or region of the human anatomy, such as a limb, an internal organ, a particular type of tissue or anatomical structure. The information provided by the subject field may later be employed to select an image for use in a subsequent comparison.

The slice-number field may be used in one or more embodiments to store information that more precisely locates the area captured in the image. For example, if human subject includes an axis running from head to toe, the slice number may indicate the distance from the top of the person's head to the location of the slice which may represent an image of a cross-section of a particular part of a subject's anatomy. Other approaches may also be employed which provide a reference system to identify a location of a slice relative to a portion of the subject's anatomy. In one embodiment, the slice-number can be used to select an image or group of adjacent images from the database for comparison with a current image.

The information provided by the size field may, for example, include the dimensions of the slice, for example, the dimensions in pixels. The dimensions may be employed to more precisely match a reference image to a subject image when performing a diagnostic comparison between the reference image and the subject image.

The ROI-location field provides information that may be employed to more precisely locate the abnormality within the image. The ROI location may be a set of coordinates or a plurality of coordinates that indicate the boundaries of the region of interest such that later comparisons with the image may take advantage of the particular information included in the region of interest.

The diagnostic-information field may provide information describing the ultimate diagnosis associated with the abnormality (e.g., pathological condition) located within the image. In some embodiments, the diagnosis information may describe the fact that the image is “normal.” That is, that the image does not represent a pathological condition.

As may be apparent from the preceding, comparisons between reference images and images submitted for diagnosis may require a certain degree of precision in correctly matching the region represented by the slice that is being evaluated for a medical diagnosis and the reference slice or slices. For example, where a particular portion of an organ is being evaluated, the reference image or images that the slices are being evaluated against should be of the same region of the organ that appears in the image undergoing evaluation. In one or more embodiments, the image records 668 provide information that facilitates a more accurate comparison.

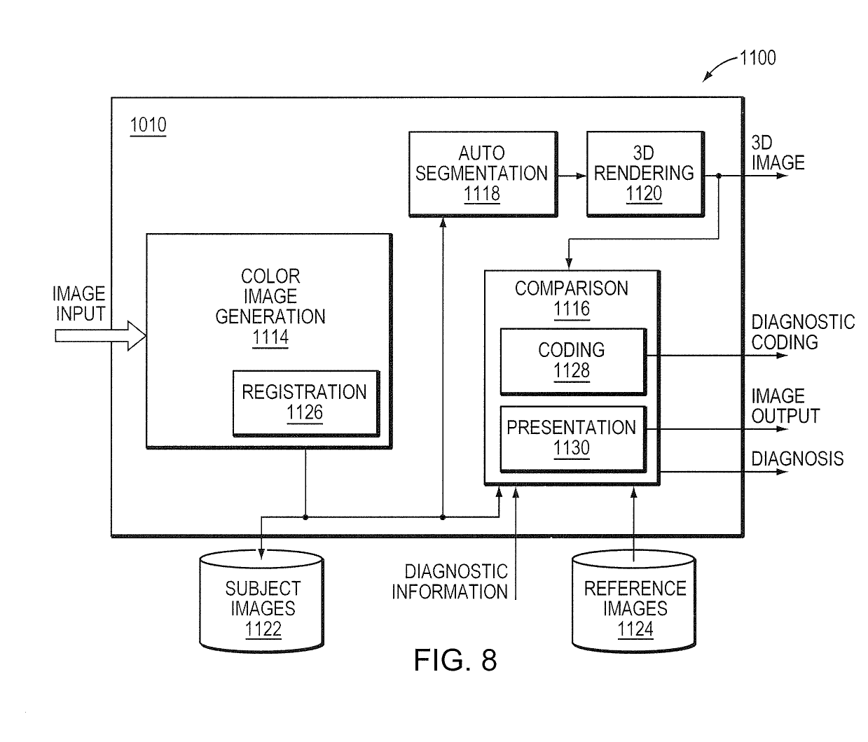

FIG. 8 illustrates an embodiment of a system 1100 for processing color MRI images with a processing module 1010 which includes a plurality of modules to perform all or some of those operations. According to one embodiment, the processing module 1010 may include a color image generation module 1114, a comparison module 1116, an auto segmentation module 1118 and a 3D rendering module 1120. The system 1100 may also include subject image storage 1122 for storing one or more subject images and reference image storage 1124 for storing one or more reference images. Further, the system 1100 may employ a variety of configurations, for example, the color image generation module 1114 may be located external to the processing module 1010. In a further embodiment, the system 1100 may receive color MRI images from an external system and/or database, and as a result, color image generation may not be included in the system 1100. Further, in the illustrated embodiment, the subject image storage 1122 and the reference image storage 1124 are included in the system 1100. In some alternate embodiments, however, either or both of the subject image storage 1122 and the reference image storage 1124 are part of an external system and are not included in the system 1100. In addition, the processing module 1010 may include a single module or a plurality of modules. Further still, where a plurality of modules are employed, they may be included in a single computer or a plurality of computers which may or may not be co-located, e.g., they may be connected over a network.

In accordance with one embodiment, the processing module 1010 receives an image input in the form of gray scale images (e.g., a series of gray scale images) and generates one or more color images (e.g., composite color images) with the color image generation module 1114. For example, in one embodiment, a plurality of sets of MRI images of an object are generated where each set employs different image parameters than others of the plurality of sets. That is, different physical attributes are highlighted in the various sets. According to one embodiment, the color image generation module 1114 operates in the manner previously described with reference to the colorization module 104 of FIG. 1 to generate composite color images from the plurality of sets of MRI images. In a further embodiment, the color image generation module 1114 includes a registration module 1126 that is adapted to spatially align the slices in each of the plurality of sets with corresponding slices in each of the others of the plurality of sets. In accordance with one embodiment, the axial coordinates along an axis of the subject (e.g., the z-axis) of corresponding slices from a plurality of sets (e.g., the sets 222, 224 and 226) are precisely aligned by referencing each set of slices to a common coordinate on the z-axis, e.g., the first slice from each set is co-located at a common starting point. In accordance with one embodiment, the registration is performed automatically, e.g., without any human intervention. In various embodiments, the distance between the slices is determined by the degree of precision required for the application. Accordingly, the axial proximity of each slice to the adjacent slices is closest where a high degree of precision is required.

In one embodiment, a first slice from a first set (e.g., image 16, set 222) is registered with a first slice from a second set (e.g., image 16, set 223) and a first slice from a third set (e.g., image 16, set 224), etc. to generate a first composite color image. A second slice from the first set (e.g., image 17, set 222) is registered with the second slice from the second set (e.g., image 17, set 223) and a second slice from the third set (e.g., image 17, set 224), etc. to generate a second composite color image. The preceding may be employed for a plurality of spatially aligned slices from each set to generate a plurality of the composite color images.

In one embodiment where the registration is performed automatically, the common coordinate is the result of a pre-processing of at least one image from each set. That is, the common coordinate may be identified by selecting an object or a part of an object that is clearly distinguishable in each set.

In general, the images generated by the color image generation module 1114 are images that provide one or more subject images that are the subject of a diagnostic analysis performed by the system 1100 and the processing module 1010. For example, a medical diagnosis may be provided as a result of an evaluation of the subject images. In one embodiment, the medical diagnosis may be accompanied by a corresponding diagnostic code and/or a confidence factor. In addition, one or more images may be generated and presented by the processing module 1010 as a result of the processing of one or more subject images.

According to one embodiment, a plurality of subject images are communicated to the auto segmentation module 1118 where, for example, one or more boundaries that appear in the subject images are more clearly defined. Further, in one embodiment, the segmentation is performed automatically, i.e., without human intervention. In a further embodiment, the results of the segmentation provide region boundaries that are accurate to within ±5 millimeters or greater accuracy without the need for post-processing, i.e., by a human reviewer.

Where the subject images include portions of the human anatomy, the segmentation may be accomplished based, at least in part, on the biological characteristics of the various regions that are represented in the images. That is, a single organ, type of tissue or other region of the anatomy may include various degrees of a plurality of biological characteristics such as a percentage of water, a percentage of fat and/or a percentage of muscle. The composite images may highlight the organ, tissue or other region as a result of these or other biological characteristics. For example, different biological characteristics and/or features may be represented by different colors, different color hues, different color intensities, other image characteristics and/or any combination of the preceding. In one embodiment, the highlighting enhances a distinction between boundaries of the various regions illustrated in the image, for example, the boundary between an organ and the body cavity where it is located.

In one embodiment, an output of the auto segmentation module 1118 is communicated to the 3D rendering module 1120 which generates a three dimensional image from the composite color images that are segmented, e.g., automatically segmented. In some embodiments, the 3D rendering module 1120 generates an improved 3D image because the segmentation provides for more clearly defined features. In one embodiment, the 3D rendering module 1120 generates a 3D image having a greater diagnostic utility than prior approaches because the composite color images are segmented. According to one embodiment, a 3D image is communicated from an output of the 3D rendering module, for example, to a display where a medical professional such as a doctor can review the 3D image. In a version of this embodiment, the 3D image is employed in a surgical planning process. According to one embodiment, the 3D image is a 3D subject image that is communicated from an output of the 3D rendering module to the comparison module 1116.

In some embodiments the 3D rendering module generates a 3D color image which may be used to model the subject, and in particular, dimensions, locations, etc. of the objects in the image (i.e., in a subject or portion thereof). The 3D image may be employed for comparison with other 3D images for medical diagnosis and/or treatment.

In other embodiments, the 3D rendering module generates a 3D image from composite color images that is not in color (e.g., it is a gray-scale or black and white image). In various embodiments, the 3D image that is not in color is employed for any of the preceding uses, for example, object location, size, comparison, etc.

In various embodiments, the comparison module 1116 is adapted to perform a comparison between one or more subject images and one or more reference images. The comparison may be performed using a single subject image, a series of related subject images (e.g., slices), or multiple series of subject images which may be compared with a single reference image, a series of related reference images (e.g., slices), or multiple series of reference images. In one embodiment, the comparison module 1116 compares a 3D subject image with a 3D reference image. In general, the comparison includes a comparison between information included in at least one subject image with information included in at least one reference image.

The reference images that are employed to perform a comparison with one or more subject images may be provided when the system 1100 issues a request, for example, to receive reference images of a certain type (e.g., a group of reference images may be selected because they include information concerning a suspect pathological condition that may be most likely to appear in the subject image or images). Further, the subject image storage 1122 need not be a database, but may instead be a RAM. That is, in one embodiment, the composite images may be temporarily stored in RAM and processed by the processor 1010, with the operations described herein on a “real-time” basis.

According to one embodiment, where the comparison is performed as part of a process for making a medical determination and/or diagnosis, the information included in the reference images is information concerning a known pathological condition. For example, the reference images may include a representation of a part of the human anatomy suffering from the pathological condition. Accordingly, the information may be in the form of a size, a shape, a color, an intensity, a hue, etc. of an object or region where the preceding characteristics provide information concerning the presence of the pathological condition.

According to one embodiment, the comparison module 1116 includes an input for receiving diagnostic information to facilitate the comparison. That is, in one embodiment, a user (e.g., a medical professional) can supply input data to focus the comparison on a certain region of the subject image and/or identify a biological characteristic/feature that is of particular importance in performing the comparison. For example, the user may indicate that the subject image(s) should be screened for a particular suspect pathological condition or a family of related pathological conditions. The user may independently or in combination with the input concerning the suspect pathological condition identify a specific part of the human anatomy that is of particular interest. Many other types of diagnostic information may be supplied to the comparison module 1116 to increase the efficiency, accuracy and/or utility of the comparison by, for example, defining some of the parameters that should be employed in the comparison.

As an additional example, the diagnostic information may include information used to establish one or more pre-determined thresholds concerning a strength of a match between subject images and reference images. In particular, the threshold may be employed to establish a maximum strength of a match where subject images with a strength of match less than the maximum are identified as not including a pathological condition or a specific pathological condition being searched for, e.g., the subject image may be identified as a “normal.” Another threshold may be employed to establish a minimum strength of a match where subject images having a strength of match greater than the minimum are considered as possibly including a pathological condition. The strength of the match may also be employed to determine a degree of confidence in the diagnosis regardless of whether the diagnosis concerns the presence of a pathological condition or an absence of a pathological condition.

According to on embodiment, the system 1100 includes a coding module 1128. That is, in one embodiment, the comparison module 1116 generates a diagnosis that one or more pathological conditions are represented in a subject image (or series of related subject images) because of, for example, the strength of the match between the subject image and one or more reference images. The coding module may employ information concerning the reference image(s), the subject image(s) or both to generate a diagnostic code corresponding to the diagnosis. For example, referring to FIG. 5, a diagnostic code “M45—Ankylosing spondylitis” appears in the display 550. In one embodiment, the information provided by the diagnostic code allows a healthcare professional to quickly interpret the results of the comparison performed by the comparison module 1116. In some embodiments, the display 550 includes a subject image or region thereof that is annotated in some fashion to highlight a suspect pathological condition that is represented in the image. For example, the image may include an outline in a geometric shape (e.g., squares, rectangles, circles etc.), pointers or other indicia that serve to more specifically identify a region within an image where the pathological condition may be represented. As previously mentioned and as also illustrated in FIG. 5, the display 550 can also include a confidence factor (i.e., “98% confidence”) corresponding to the diagnosis.

In accordance with one embodiment, the system 1100 includes a presentation module. According to the embodiment illustrated in FIG. 8, a presentation module 1130 is included in the comparison module 1116 and generates an image output for display. In other embodiments, however, the presentation module 1116 is included elsewhere in the processing module 1010 or elsewhere in the system 1100. For example, in one embodiment, the presentation module is included in the processor 1010 outside the comparison module 1116 and is employed to generate any or all of 3D image outputs, other image outputs, diagnosis information, and diagnostic coding information for display, i.e., for display in the display 114 at the user interface 112.

In one embodiment, all or a portion of the processing module 1010 is a software-based system. That is, the processing module 1010 including any one or any combination of the color image generation module 1114, the registration module 1126, the auto-segmentation module 1118, the 3D rendering module 1120, the comparison module 1116, the coding module 1128 and the presentation module 1130 may be implemented in any of software (e.g., image processing software), firmware, hardware or a combination of any of the preceding. According to one embodiment, the processing module is included in a computer.

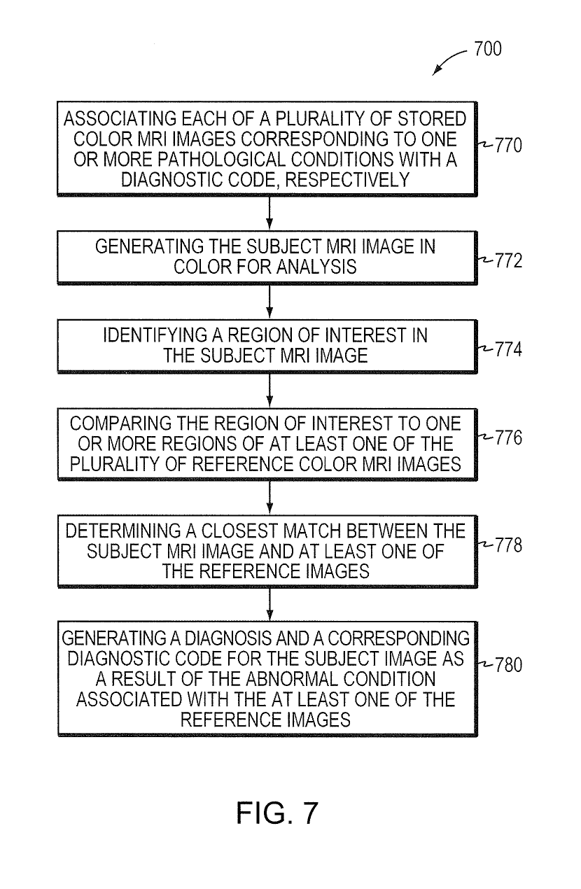

Referring now to FIG. 7, a process 700 for assigning a diagnostic code to a subject MRI image is shown in accordance with one embodiment. In one version, the process 700 is employed with the system 100 illustrated in FIG. 1. In various alternative embodiments, the process 700 may be performed using various alternate systems that include a processing module. In accordance with one embodiment, the process 700 begins at act 770 where each of a plurality of reference color MRI images corresponding to one or more abnormal pathological conditions is associated with a diagnostic code. In one embodiment, the diagnostic code identifies the specific pathological condition or conditions that appears in the associated reference image. Thus, a plurality of reference images may include a plurality of different diagnostic codes.

At act 772, a subject MRI image is generated in color for analysis. The colorization included in act 772 may be achieved using any of the previously referenced processes. Further, in some embodiments, the act 772 is not included in the process 700. Instead, in some embodiments, the subject image is generated in color in an independent process. At act 774, a region of interest is identified in the subject MRI image. The identification of the region of interest may occur automatically or alternatively may be identified manually by a health care professional.

At act 776, one or more reference images is retrieved from the image database and the subject image is compared to each of the reference images that are retrieved. In accordance with one embodiment, the identification and retrieval of the reference image or images, at act 776, is the result of information included in the image record that, for example, identifies a subject and/or slice number (or a plurality of slice numbers) that is relevant to the image undergoing analysis.

At act 778, the closest matching image between the subject MRI image and the reference images that are retrieved is identified. In accordance with one embodiment, the closest match is the result of a comparison of both color, hue and intensity appearing in the region of interest identified in the subject image and a region of interest in the closest reference image.

At stage 780, a diagnosis is generated for the subject image as a result of the diagnosis information associated with the reference image. In one embodiment, the diagnosis identifies a pathological condition. In a further embodiment, a diagnosis code is generated which corresponds to the diagnosis generated at act 780.

A general-purpose computer system (e.g., the computer 116) may be configured to perform any of the described functions including but not limited to generating color MRI images, automatically segmenting a plurality of color images, generating a 3D color MRI image, performing diagnostic comparisons using one or more subject images and one or more reference images and communicating any of a diagnosis, a diagnostic code and color MRI images to a user interface. It should be appreciated that the system may perform other functions, including network communication, and the invention is not limited to having any particular function or set of functions.

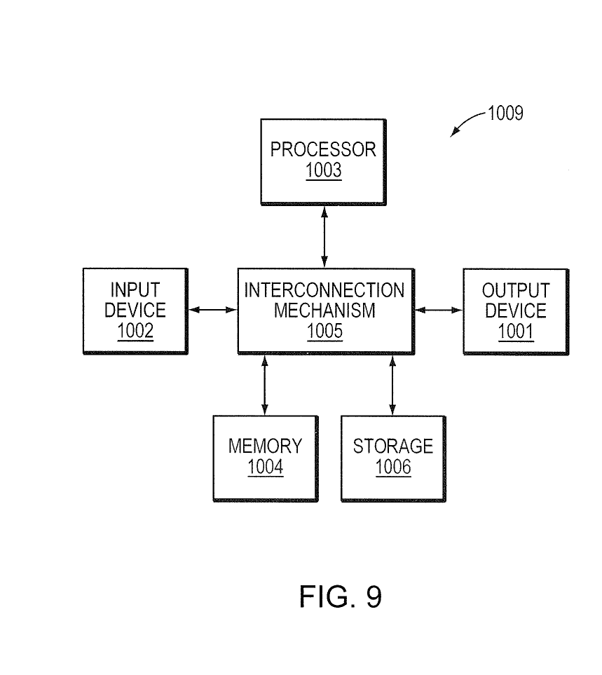

For example, various aspects of the invention may be implemented as specialized software executing in a general-purpose computer system 1009 (e.g., the computer 116) such as that shown in FIG. 9. The computer system 1009 may include a processor 1003 or a plurality of processors connected to one or more memory devices 1004, such as a disk drive, memory, or other device for storing data. Memory 1004 is typically used for storing programs and data during operation of the computer system 1009. Components of computer system 1009 may be coupled by an interconnection mechanism 1005, which may include one or more busses (e.g., between components that are integrated within a same machine) and/or a network (e.g., between components that reside on separate discrete machines). The interconnection mechanism 1005 enables communications (e.g., data, instructions) to be exchanged between system components of system 1009.

Computer system 1009 also includes one or more input devices 1002, for example, a keyboard, mouse, trackball, microphone, touch screen, and one or more output devices 1001, for example, a printing device, display screen, speaker. In addition, computer system 1009 may contain one or more interfaces (not shown) that connect computer system 1009 to a communication network (in addition or as an alternative to the interconnection mechanism 1001.

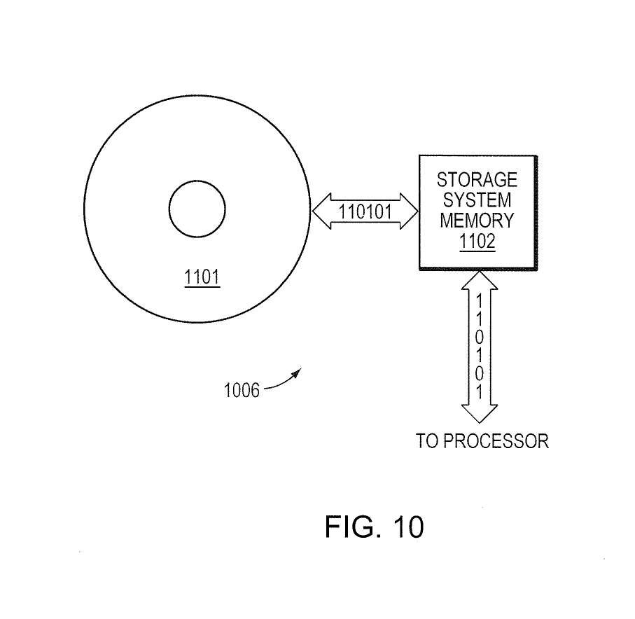

The storage system 1006, shown in greater detail in FIG. 10, typically includes a computer readable and writeable nonvolatile recording medium 1101 in which signals are stored that define a program to be executed by the processor or information stored on or in the medium 1101 to be processed by the program. The medium may, for example, be a disk or flash memory. Typically, in operation, the processor causes data to be read from the nonvolatile recording medium 1101 into another memory 1102 that allows for faster access to the information by the processor than does the medium 1101. This memory 1102 is typically a volatile, random access memory such as a dynamic random access memory (DRAM) or static memory (SRAM). It may be located in storage system 1006, as shown, or in memory system 1004, not shown. The processor 1003 generally manipulates the data within the integrated circuit memory 1004, 1102 and then copies the data to the medium 1101 after processing is completed. A variety of mechanisms are known for managing data movement between the medium 1101 and the integrated circuit memory element 1004, 1102, and the invention is not limited thereto. The invention is not limited to a particular memory system 1004 or storage system 1006.

The computer system may include specially-programmed, special-purpose hardware, for example, an application-specific integrated circuit (ASIC). Aspects of the invention may be implemented in software, hardware or firmware, or any combination thereof. Further, such methods, acts, systems, system elements and components thereof may be implemented as part of the computer system described above or as an independent component.

Although computer system 1009 is shown by way of example as one type of computer system upon which various aspects of the invention may be practiced, it should be appreciated that aspects of the invention are not limited to being implemented on the computer system as shown in FIG. 9. Various aspects of the invention may be practiced on one or more computers having a different architecture or components that that shown in FIG. 9.

Computer system 1009 may be a general-purpose computer system that is programmable using a high-level computer programming language. Computer system 1009 may be also implemented using specially programmed, special purpose hardware. In computer system 1009, processor 1003 is typically a commercially available processor such as the well-known Pentium class processor available from the Intel Corporation. Many other processors are available. Such a processor usually executes an operating system which may be, for example, the Windows 95, Windows 98, Windows NT, Windows 2000 (Windows ME) or Windows XP operating systems available from the Microsoft Corporation, MAC OS System X operating system available from Apple Computer, the Solaris operating system available from Sun Microsystems, or UNIX operating systems available from various sources. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

One or more portions of the computer system may be distributed across one or more computer systems coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects of the invention may be performed on a client-server or multi-tier system that includes components distributed among one or more server systems that perform various functions according to various embodiments of the invention. These components may be executable, intermediate (e.g., IL) or interpreted (e.g., Java) code which communicate over a communication network (e.g., the Internet) using a communication protocol (e.g., TCP/IP).

It should be appreciated that the invention is not limited to executing on any particular system or group of systems. Also, it should be appreciated that the invention is not limited to any particular distributed architecture, network, or communication protocol.

Various embodiments of the present invention may be programmed using an object-oriented programming language, such as SmallTalk, Java, C++, Ada, or C# (C-Sharp). Other object-oriented programming languages may also be used. Alternatively, functional, scripting, and/or logical programming languages may be used. Various aspects of the invention may be implemented in a non-programmed environment (e.g., documents created in HTML, XML or other format that, when viewed in a window of a browser program, render aspects of a graphical-user interface (GUI) or perform other functions). Various aspects of the invention may be implemented as programmed or non-programmed elements, or any combination thereof.

The process 1000 and the various acts included therein and various embodiments and variations of these acts, individually or in combination, may be defined by computer-readable signals tangibly embodied on a computer-readable medium for example, a non-volatile recording medium in integrated circuit memory element or a combination thereof. Such signals may define instructions, for example as part of one or more programs, that, as a result of being executed by a computer instruct the computer to perform one or more of the methods or acts described herein, and/or various embodiments, variations and combinations thereof. The computer-readable medium on which such instructions are stored may reside on one or more of the components of the system 1009 described above, and may be distributed across one or more of such components.

The computer-readable medium may be transportable such that the instructions stored thereon can be loaded onto any computer system resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement the above discussed aspects of the present invention.

The computer described herein may be a desktop computer, a notebook computer, a laptop computer, a handheld computer or other computer that includes a control module to format one or more inputs into an encoded output signal. In particular, the computer can include any processing module (e.g., the processing module 1010) that can be employed to perform a diagnostic analysis of a subject image.

Although the methods and systems thus far described are placed in the context of the health care field, and in particular, performing a medical diagnosis, surgical planning, etc. embodiments of the invention may also be employed in any other fields in which color MRI images are used including non-medical uses. For example, embodiments of the invention may be used in the fields of food and agricultural science, material science, chemical engineering, physics and chemistry. Further, various embodiments, may be employed to improve guidance in surgical robotic applications.

Embodiments of the invention, may also be employed in multi-modal imaging and diagnostic systems (i.e., systems in which an image generated via a first imaging technology (e.g., MRI) is overlayed with an image generated via a second imaging technology (e.g., CT scan).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of automatically generating a diagnosis based on information provided in a subject MRI image, the method comprising acts of:
associating each of a plurality of reference color MRI images corresponding to one or more pathological conditions with a diagnosis, respectively;
identifying a region of interest in the subject MRI image;
comparing the region of interest to one or more regions of at least one of the plurality of reference color MRI images;
determining a closest match between the subject MRI image and a reference image selected from among the plurality of reference color MRI images; and
generating a diagnosis associated with the subject image based at least partly on a pathological condition associated with the reference image.

2. The method of claim 1, further comprising an act of assigning a confidence factor to the diagnosis.

3. The method of claim 1, further comprising an act of assigning a diagnosis code to the subject image, wherein the diagnosis code corresponds to the generated diagnosis.

4. The method of claim 3, wherein the act of generating the diagnosis includes an act of generating a diagnosis that at least one color MRI image is normal.

5. The method of claim 4, further comprising an act of determining whether at least one feature of a pathological condition is represented in the color MRI image and generating a diagnosis that the at least one color image is normal when the pathological condition is not present.

6. The method of claim 4, further comprising an act of assigning a confidence factor to the diagnosis that the at least one color MRI image is normal.

7. The method of claim 1, wherein the act of generating the subject MRI includes an act of automatically generating the subject MRI based on both pixel intensities occurring in at least one gray-scale image and an image-generating parameter with which the at least one gray-scale image is generated.

8. The method of claim 1, further comprising an act of determining a strength of the closest match.

9. The method of claim 8, wherein the act of determining the strength of the closest match includes an act of determining an area of a particular type of tissue in the region of interest.

10. The method of claim 1, wherein the act of identifying further comprises an act of automatically identifying the region of interest.

11. The method of claim 1, further comprising an act of identifying the region of interest from among a plurality of regions automatically selected.

12. The method of claim 1, further comprising an act of generating the diagnosis based on information provided in a plurality of subject MRI images.

13. The method of claim 12, further comprising an act of generating the plurality of subject MRI images as a series of color slices of a subject.

14. The method of claim 12, further comprising acts of:
identifying a region of interest in each of the plurality of subject MRI images; and
comparing each region of interest with one or more regions of at least one of the plurality of reference MRI images.

15. The method of claim 14, further comprising an act of determining a closest match between each of the plurality of subject images and a reference image selected from among the plurality of reference MRI images.

16. The method of claim 15, further comprising an act of employing a scale to spatially align each of the plurality of subject images with at least one of the reference images.

17. The method of claim 15, wherein the act of determining further includes an act of, for each of the plurality of subject images, individually selecting the subject image from the plurality of subject images and selecting the reference image based on information in the selected subject image.

18. A system for generating a diagnostic code based on information provided in a subject MRI image, the system comprising:
a reference image storage module configured to store a plurality of color reference MRI images that include at least one reference image having a region in which a known pathological condition is represented;
a processing module configured to compare the subject image with the at least one reference image; and
a coding module configured to generate a diagnostic code concerning the subject image based on a comparison between the information provided in the subject MRI image and information provided in the at least one reference image.

19. The system of claim 18, wherein the information provided in the at least one reference image is information concerning the pathological condition.

20. The system of claim 19, wherein the information concerning the pathological condition is information concerning the region in which the known pathological condition is represented.

21. The system of claim 20, wherein the information concerning the pathological condition is provided by color that is present in the region.

22. The system of claim 21, wherein the information concerning the pathological condition is provided by a plurality of colors that are present in the region.

23. The system of claim 20, wherein the information concerning the pathological condition is provided by an intensity of at least one color that is present in the region.

24. The system of claim 20, wherein the information concerning the pathological condition is provided by a hue of at least one color that is present in the region.

25. The system of claim 18, wherein the processing module is further configured to determine a strength of a match between the subject image and the at least one reference image.

26. The system of claim 25, wherein the diagnostic code is based, at least in part, on the strength of the match.

27. The system of claim 25, wherein the processing module is further configured to compare the subject image with a plurality of reference images.

28. The system of claim 27, wherein the coding module is further configured to generate the diagnostic code based on a comparison between the information provided in the subject MRI image and information provide in a plurality of reference images.

29. The system of claim 18, wherein the processing module is further configured to compare a plurality of subject images with a plurality of reference images.

30. The system of claim 29, wherein the processing module is further configured to compare each of the plurality of subject images to the plurality of reference images and to independently determine a strength of a match of each of the plurality of subject images and at least one of the plurality of reference images.

31. The system of claim 30, wherein the processing module is further configured to determine a closest match from among those comparisons for which the strength of the match is determined.

32. The system of claim 18, further comprising a colorization module configured to generate the subject MRI image in color by generating a composite color image from a plurality of gray-scale images.
